(19) United States
(12) Patent Application Publication
(10) Pub. No.: US 2026/0317672 A1
(43) Pub. Date: Oct. 15, 2026

(54) HOLDING DEVICE FOR A CONTAINER, HAVING A MAGNETIC DRIVE FOR A SEPARATE AGITATOR SHAFT

(71) Applicant: Sartorius Stedim Biotech GmbH, Goettingen (DE)

(72) Inventors: Thomas REGEN, Goettingen (DE); Sandra BURGHARDT, Goettingen (DE); Simon TOPP-MANSKE, Guxhagen (DE); Ute HUSEMANN, Goettingen (DE)

(21) Appl. No.: 19/116,528

(22) PCT Filed: Oct. 4, 2023
(86) PCT No.: PCT/EP2023/077390

§ 371 Date: Mar. 28, 2025

(30) Foreign Application Priority Data
Oct. 6, 2022 (EP) 22200127.3

(51) Int. Cl. C12M 1/06; B01F 27/213; B01F 35/31; C12M 1/00

(52) U.S. Cl. CPC C12M 27/02; B01F 27/213; B01F 35/31; C12M 23/26; C12M 23/28

(57) ABSTRACT

A holding device for a container, in particular a bioreactor, including a magnetic drive for a separate stirrer shaft and a lever mechanism for releasing the stirrer shaft from the magnetic drive. The magnetic drive has a first coupling side and the stirrer shaft has a second coupling side of a magnetic coupling, which can be connected to the first coupling side. The lever mechanism has at least one pressure section and can be pivoted between a closed position and a release position. When the lever mechanism is pivoted to the release position, the pressure section is moved downwards to engage directly or indirectly on the stirrer shaft.

### FIELD OF THE INVENTION

The invention relates to a holding device for a container, in particular a bioreactor, having a magnetic drive for a separate stirrer shaft.

### BACKGROUND

Single-use bioreactors, which are formed from a flexible bag and the contents of which are to be mixed using a stirrer having a rotatable stirrer shaft, are typically received in a stable holding device in which a drive for the stirrer shaft is also provided. The power transmission between the drive, which is arranged outside the bioreactor, and the stirrer shaft, which is arranged inside the bioreactor, can be carried out in a known manner by means of a magnetic coupling. To close the coupling, i.e. to magnetically connect the stirrer shaft to the magnetic drive, the stirrer shaft must be manually lifted and positioned at a sufficiently small distance below the magnet of the drive. For the subsequent release of the coupling, the stirrer shaft is manually pushed away downwards. This process usually takes place in an uncontrolled manner, and handling is sometimes very difficult due to the strong magnetic forces.

### SUMMARY

The object of the invention is to enable a magnetic clutch, by means of which a stirrer shaft is coupled to a magnetic drive, to be released in a controlled and safe manner.

This object is achieved by a holding device having the features of claim 1. Advantageous and functional configurations of the holding device according to the invention are specified in the sub-claims.

The holding device according to the invention for a container, in particular a bioreactor, comprises a magnetic drive for a separate stirrer shaft and a lever means for releasing the stirrer shaft from the magnetic drive. The magnetic drive has a first coupling side and the stirrer shaft has a second coupling side of a magnetic coupling, which can be connected to the first coupling side. The lever means has at least one pressure section and is pivotable between a closed position and a release position. When the lever means is pivoted into the release position, the pressure section is moved downwards to engage directly or indirectly on the stirrer shaft.

The invention is based on the finding that, in a bioreactor having a stirrer shaft, the magnetic attraction force of a closed magnetic coupling between the magnetic drive and the stirrer shaft may be very large, but also decreases very strongly with increasing distance. Normally, the magnetic force of the magnetic coupling is dependent on the required torque and increases with the size of the bioreactor and the specified maximum speed. The forces are typically between 50 and 600 N. It is difficult for a single operator to overcome a force of this magnitude in a controlled manner, especially since manually gripping the stirrer shaft is also cumbersome.

The lever means of the holding device according to the invention allows the forces to be overcome to be significantly reduced due to the lever effect. Due to the magnetic force decreasing greatly with distance—as a rough approximation, the magnetic force is approximately inversely proportional to the square of the distance—the lever means only has to ensure that a distance of a few centimeters is created between the magnetic drive and the stirrer shaft. The magnetic force is then already so weak that an operator can easily hold and remove the bioreactor. At the same time, the lever means allows for considerably simplified handling, since the operator does not have to pull on the stirrer shaft, but can instead conveniently push a lever downwards.

Not only the removal, but also the mounting of the stirrer shaft to the magnetic drive can be carried out in an easier and more controllable manner by means of the lever means of the holding device according to the invention. The lever means can be used to prevent the magnetic coupling from closing prematurely during the positioning of the stirrer shaft under the magnetic drive, where correct positioning has not yet been achieved or a hand of the operator could be crushed. This means that the operator can calmly position the stirrer shaft correctly without the stirrer shaft coming so close to the magnetic drive that the magnetic force could interfere with the handling and, in particular, the positioning.

Both the mounting and removal of the stirrer shaft from the magnetic drive may be accomplished by a single operator due to the lever means, the risk of injury to the operator or damage to the bioreactor being largely excluded.

According to a preferred embodiment of the invention, the lever means includes an adapter plate having a lever pivotally mounted thereon and a central opening which is located opposite the first coupling side and through which the second coupling side of the stirrer shaft projects in the mounted state of the stirrer shaft. The design of the adapter plate, which can be stably mounted in the holding device, can be adapted to the structural conditions of the holding device, in particular of the magnetic drive, and serves as a bearing for the pivotable lever of the lever means.

Preferably, the lever means is detachably fastened to the holding device by at least one fastening section. For example, special fastening sections adapted to the structural conditions may be provided, by means of which the lever means can be installed and uninstalled again. The detachable fastening also allows the lever means to be used on other holding devices.

The use of a plurality of pressure sections arranged in a distributed manner allows the forces generated by the lever movement to be distributed evenly over the upper end of the stirrer shaft, so that the release of the stirrer shaft from the magnetic drive is not accompanied by an uncontrolled tilting movement.

The forces introduced by the pivoting movement of the lever should act on the stirrer shaft as effectively as possible so that it can be easily released from the magnetic drive. In a preferred design of the lever means, it is provided that the at least one pressure section is formed by a linearly guided sliding element which moves vertically downwards when the lever means pivots to the release position. This ensures that the forces introduced act precisely in the direction in which the stirrer shaft is released from the magnetic drive.

A conversion of the pivoting movement of the lever into a linear movement of the at least one sliding element can be achieved by the sliding element being rotatably mounted on a pivot section of the lever means and guided in a recess of the adapter plate.

According to a further development of the invention, the lever means has a securing mechanism which can be activated and deactivated to increase safety during operation of the stirrer shaft coupled to the magnetic drive. When the securing mechanism is activated, the lever means is fixed in the closed position, whereas when the securing mechanism is deactivated, the lever means can be pivoted between the closed position and the release position. Activating the securing mechanism after the stirrer shaft has been coupled ensures that the lever means cannot be pivoted into the release position unintentionally during operation.

Furthermore, the lever means may also include a release-position securing mechanism which can be activated and deactivated and which, in the activated state, fixes the lever means in the release position, whereas, in the deactivated state, it allows the lever means to pivot between the release position and the closed position.

According to a particular aspect of the invention, the lever means may be supplemented by a separate gripper. The gripper is shaped such that it can be mounted on a collar of the stirrer shaft. This makes the stirrer shaft easier to handle. The gripper also has at least one counterpressure section having a counterpressure surface which faces upwards in the mounted state of the stirrer shaft and comes into engagement with the pressure section when the lever means is pivoted to the release position. The application of force via the counterpressure surface(s) of the gripper prevents the upper end of the stirrer shaft from coming into direct contact with the pressure section(s) of the lever means, so that the container, in particular the flexible bioreactor bag, is not (accidentally) damaged during removal. In addition, the gripper may serve to bridge a distance between the lever means and the container and to provide a flat surface on the connection geometry.

The gripper preferably has a circular or partially circular receiving section having an inner diameter which corresponds approximately to an outer diameter of the first coupling side of the magnetic drive. Due to the matching of the diameters, the gripper along with the stirrer shaft can be easily positioned under the magnetic drive such that the two coupling sides are exactly opposite each other. The receiving section then engages around the downwardly-projecting first coupling side of the magnetic drive. This guides the movement of the stirrer shaft caused by the magnetic force towards the magnetic drive, and prevents a hand or finger of the operator from accidentally getting caught between the two coupling sides.

A particularly convenient handling is allowed by designing the gripper with at least one gripping section, by which an operator can simply hold the gripper.

In the case of a very large container, handling may be facilitated by a transport device for lifting and transporting the container, as is basically known, for example, from DE 10 2013 002 091 B3. The gripper may be part of such a transport device, so that the operator does not have to hold and transport the container himself before assembly or after disassembly.

Preferably, the gripper may be detachably mounted on the stirrer shaft so that the gripper can be removed again after the stirrer shaft has been coupled and can be used for other purposes if necessary.

At least part of the lever means (certain components) of the holding device according to the invention can be manufactured by an additive manufacturing method. In particular, these components of the lever means and, if necessary, other components of the holding device can be manufactured by selective laser sintering (SLS) from a powdered starting material such as polyamide. In general, manufacturing using an additive manufacturing process has the advantage that, in terms of the design of the internal geometries, the material loss and the assembly costs are lower compared to conventional manufacturing processes. This is particularly relevant for the embodiments in which complex geometries or geometries which are otherwise difficult to realize are provided, such as slot geometries or cutouts for slot nuts. A further advantage may be seen in the fact that additive manufacturing methods offer the possibility of realizing large radii, for example to protect the flexible bioreactor bag from damage by edges.

It may possibly be difficult for the operator to manually operate the lever means, for example if the container is very large and the handle of the lever means is therefore not readily accessible. Therefore, a particular embodiment of the invention provides an actuation means coupled to the lever means for remote actuation of the lever means. This means that the actuation means can be used to pivot the lever means between the closed position and the release position and/or between the release position and the closed position.

The invention also provides an arrangement comprising a holding device, as defined above, and a container, in particular a bioreactor, comprising a stirrer shaft arranged inside the container and having a second coupling side (matching the first coupling side of the magnetic drive) at its upper end. The stirrer shaft is mounted on the magnetic drive of the holding device by closing the magnetic coupling such that the magnetic drive can set the stirrer shaft in a rotary motion.

### BRIEF DESCRIPTION OF THE DRAWINGS

Further features and advantages of the invention will become apparent from the following description and the accompanying drawings, to which reference is made and in which:

FIG. 1a shows a perspective view of a lever means for a bioreactor holding device according to the invention in an upper closed position according to a first variant of a first embodiment;

FIG. 1b shows a perspective view of the lever means of FIG. 1a in a release position;

FIG. 2 shows a front view of the lever means of FIG. 1a in the release position;

FIG. 3a shows a perspective view of a lever means for a bioreactor holding device according to the invention in an upper closed position according to a second variant of the first embodiment;

FIG. 3b shows a perspective view of the lever means of FIG. 3a in the fixed release position;

FIG. 4 shows a perspective view of the bioreactor holding device according to the invention in the first embodiment with a magnetic drive, a stirrer shaft coupled to the magnetic drive and a lever means in the upper closed position;

FIG. 5 shows a front view of the holding device of FIG. 4;

FIG. 6 shows a perspective view of the holding device of FIG. 4 when releasing the magnetic coupling;

FIG. 7 shows a front view of the holding device of FIG. 4 when releasing the magnetic coupling;

FIG. 8 shows a perspective view of the holding device of FIG. 4 with the gripper removed;

FIG. 9 shows a perspective view of a lever means for a bioreactor holding device according to the invention in a first variant of a second embodiment;

FIG. 10 shows a further perspective view of the lever means of FIG. 9;

FIG. 11 shows a perspective view of a lever means for a bioreactor holding device according to the invention in a second variant of the second embodiment;

FIG. 12 shows a further perspective view of the lever means of FIG. 11;

FIG. 13 shows a perspective view of part of the lever means according to the second embodiment;

FIG. 14 shows a perspective view of the adapter plate of the lever means according to the second embodiment;

FIG. 15 shows a perspective view of the pressure ring of the lever means according to the second embodiment;

FIG. 16 shows a perspective view of one of the slot nuts of the lever means according to the second embodiment;

FIG. 17 shows a perspective view of the locking ring of the lever means according to the second embodiment;

FIG. 18 shows a perspective view of the supporting ring of the lever means according to the second embodiment;

FIG. 19 shows a perspective view of the lever (without the gripping piece) according to the first variant and the lever according to the second variant of the second embodiment;

FIG. 20 shows a perspective view of the gripping piece of the lever means according to the second variant of the second embodiment;

FIG. 21 shows a perspective view of connecting elements of the lever means according to the second embodiment;

FIG. 22 shows a perspective view of one of the screw-on counter surfaces of the lever means according to the second embodiment;

FIG. 23 shows a perspective view of the adapter plate counterpart of the lever means according to the second embodiment;

FIG. 24 shows a perspective sectional view of a detail of the lever means with the latched adapter plate counterpart according to the second embodiment in the upper closed position; and

FIG. 25 shows a perspective view of a transport device for lifting and transporting a container, in particular a bioreactor, with a gripper.

### DETAILED DESCRIPTION

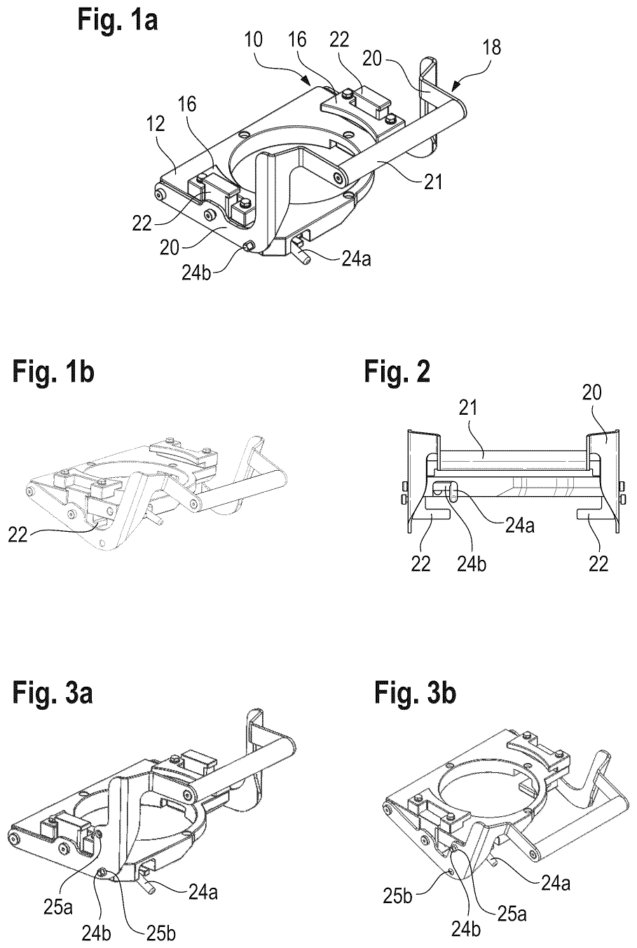

FIGS. 1a, 1b and 2 each separately show a first variant of a first embodiment of a lever means 10 for separating a stirrer shaft from a magnetic drive in an upper closed position (FIG. 1a) and in a release position (FIGS. 1b and 2). The lever means 10 comprises an adapter plate 12 having a central opening 14 through which the upper end of a stirrer shaft can project.

The adapter plate 12 has fastening sections 16 by means of which the adapter plate 12 can be fastened to a holding device for a container, in this case a single-use bioreactor. In the illustrated example embodiment, the fastening sections 16 are formed by screw-on clamping jaws which provide counter surfaces for mounting on the holding device.

A lever 18 is pivotally mounted on the adapter plate 12. In the illustrated example embodiment, the lever 18 is formed by two pivot sections 20, the front ends of which are connected to each other by a handle 21. The rear ends of the pivot sections 20 are rotatably mounted on the rear end of the adapter plate 12 on the side surfaces thereof.

The lever 18 also comprises pressure sections 22, which are moved downwards when the lever 18 is actuated. In the illustrated example embodiment, the pressure sections 22 are formed by two sliding elements which are guided in recesses in the adapter plate 12. The pressure sections 22 are rotatably mounted on the pivot sections 20 so that when the lever 18 pivots, the pressure sections 22 move linearly and vertically downwards due to the guide in the recesses of the adapter plate 12 and thus come out of the adapter plate 12, as can be seen in FIGS. 1b and 2.

Furthermore, the lever means 10 has a closed-position securing mechanism. When the closed-position securing mechanism is activated, the lever 18 is blocked in the upper closed position shown in FIG. 1a, i.e. a pivoting movement of the lever 18 into the release position shown in FIGS. 1b and 2 is not possible. If the closed-position securing mechanism is deactivated, the lever 18 can be moved back and forth between the release position and the upper closed position.

In particular, the release position can be specified in a defined manner by a stop or the like which limits the extent of the downward pivoting movement.

The lever means 10 can also be fixed in the release position (lower closed position). By means of a release-position securing mechanism, the lever 18 can be blocked in the release position shown in FIGS. 1b and 2 so that the lever 18 cannot be pivoted upwards. Of course, the release-position securing mechanism can also be deactivated again manually to allow the lever 18 to pivot between the release position and the upper closed position.

A specific, but still only exemplary design of the closed-position and release-position securing mechanism is briefly explained on the basis of the second variant of the lever means 10 shown in FIGS. 2a and 2b.

A securing pin 24a which can be fixed in two positions is connected to a securing bolt 24b which can be moved axially in or on the adapter plate 12 and in turn can interact with two bores 25a, 25b in the pivot sections of the lever 18.

To fix the lever in the upper closed position, the operator pushes the securing pin 24a to the left (according to the illustration in the figures) so that the securing bolt 24b penetrates the lower bore 25b which is aligned therewith and prevents a pivoting movement of the lever 18. Rotating the securing bolt 24b downwards about the longitudinal axis thereof fixes the securing bolt 24b in this position, and the closed-position securing mechanism is activated.

The lever 18 can also be fixed when it is in the lower release position. For this purpose, the securing bolt 24b is pushed by the operator through the upper bore 25a, which is aligned therewith in this position, by means of the securing pin 24a and fixed by turning it downwards, as a result of which the release-position securing mechanism is activated.

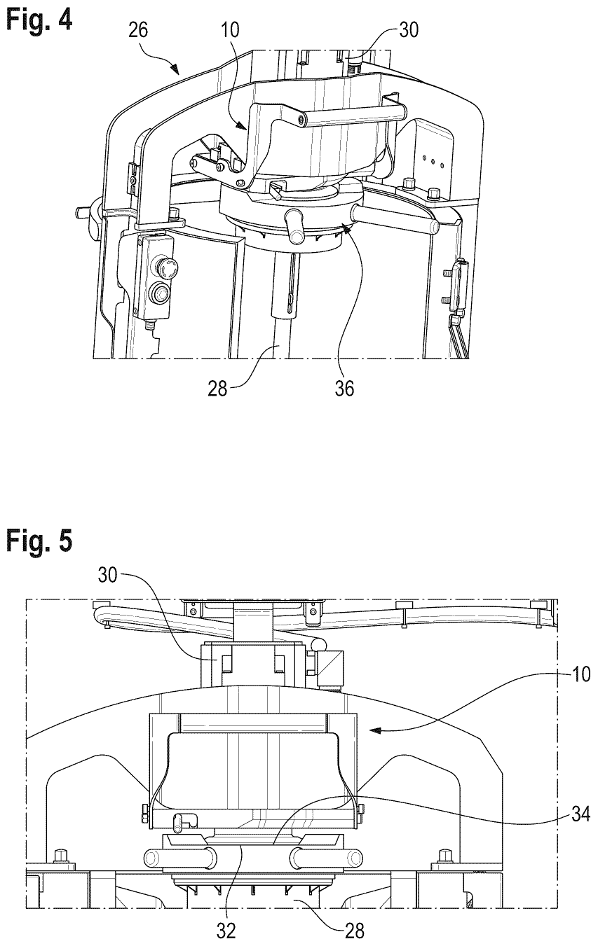

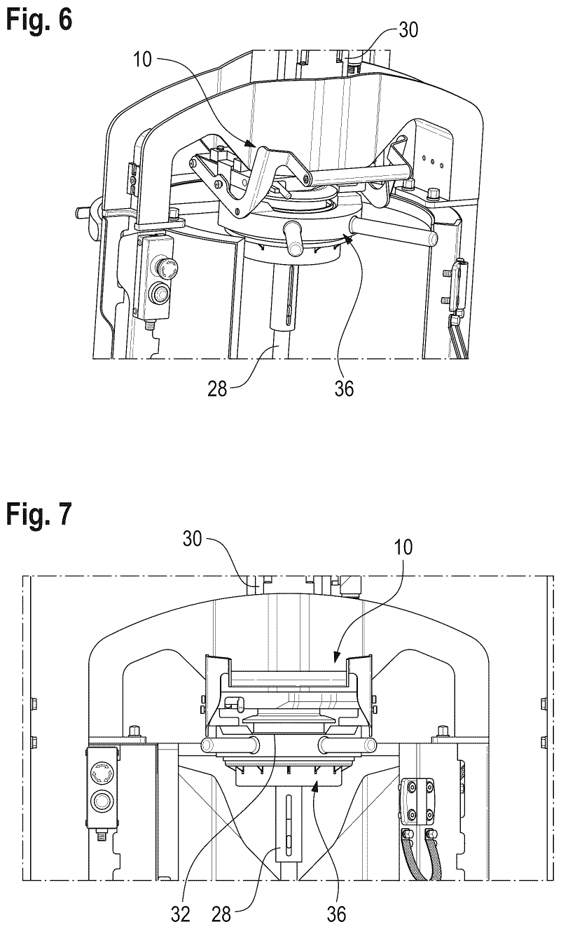

The use of the lever means 10 in a holding device 26 for a single-use bioreactor, both of which are part of a bioreactor system, is described in the following with reference to FIGS. 4 to 8.

A bioreactor (not shown separately in the figures) with a stirrer shaft 28 arranged therein is to be mounted in the holding device 26 such that a magnetic drive 30 of the holding device 26 can magnetically set the stirrer shaft 28 in a rotary motion without there being a direct mechanical connection between the drive 30 and the stirrer shaft 28. The bioreactor is mounted in the holding device 26 by means of a magnetic coupling. The magnetic drive 30 has a circular, downwardly projecting magnetic first coupling side 32. A matching, upwardly facing second coupling side 34 is formed at the upper end of the stirrer shaft 28.

The lever means 10 is fastened to the holding device 26 by means of the fastening sections 16 such that the opening 14 in the adapter plate 12 is opposite the first coupling side 32 of the drive 30, so that the first coupling side 32 is accessible from below. The lever means 10 is preferably detachably fastened so that it can be removed from the holding device 26 and fastened to another holding device.

To mount the bioreactor with the stirrer shaft 28 in the holding device 26, the magnetic coupling between the magnetic drive 30 and the stirrer shaft 28 must be closed. To this end, the stirrer shaft 28 in the bioreactor is moved towards the magnetic drive 30 so that the two coupling sides 32, 34 face each other.

A gripper 36 can be used to facilitate handling and to simplify the positioning of the bioreactor with the stirrer shaft 28. The gripper 36 has a receiving section 38, counterpressure sections with upwardly facing counterpressure surfaces 40 and laterally outwardly projecting gripping sections 42. The receiving section 38 of the gripper 36 is approximately semi-circular and adapted to receive a collar at the upper end of the stirrer shaft 28 so that the gripper 36 is stably but detachably mounted on the stirrer shaft 28. The gripping sections 42 then allow an operator to hold and position the entire bioreactor with the stirrer shaft 28.

The receiving section 38 of the gripper 36 is also adapted to the downwardly projecting first coupling side 32 of the magnetic drive 30. More specifically, the inner diameter of the receiving section 38 corresponds approximately to the outer diameter of the first coupling side 32 of the magnetic drive 30.

When the gripper 36 is used, the bioreactor is mounted in the holding device 26 as follows: the operator presses the lever 18 of the lever means 10 downwards until it is in the release position and activates the release-position securing mechanism so that the lever 18 is automatically held securely in this position. (Even without such a release-position securing mechanism, the lever 18 would remain in the release position after overcoming the magnetic holding force, but it could be accidentally pushed upwards again.) While the lever means 10 is in the release position, the operator positions the bioreactor under the magnetic drive 30 by means of the gripper 36 so that the two coupling sides 32, 34 are opposite each other. The downwardly projecting pressure sections 22 of the lever means 10 hold the upper end of the stirrer shaft 28 at a distance and prevent premature closing of the magnetic coupling. The pressure sections of the lever means 10 then press against the counterpressure surfaces 40 of the gripper 36.

After the operator has positioned the receiving section 38 of the gripper 36 around the downwardly projecting first coupling side 32 of the magnetic drive 30, he first releases the release-position securing mechanism. Thereafter, he allows the lever 18 of the lever means 10 to pivot upwards to the upper closed position, so that the pressure sections 22 move upward and no longer project from the adapter plate 12. Attracted by the magnetic force of the first coupling side 32, the upper end of the stirrer shaft 28 moves upwards until the magnetic coupling is closed (see FIGS. 4 and 5). This movement is guided by the interaction of the receiving section 38 of the gripper 36 with the protruding first coupling side 32.

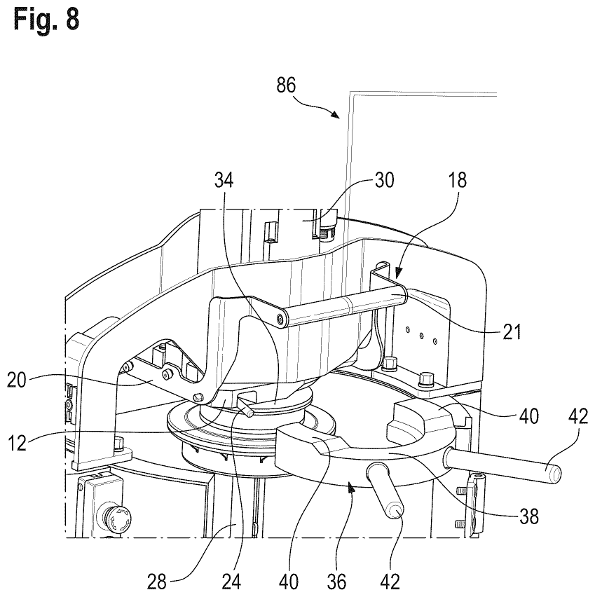

After the bioreactor has been mounted in the holding device 26, the gripper 36 can be removed from the stirrer shaft 28 (see FIG. 8).

If the mounting is performed without a gripper 36, the operator must position the stirrer shaft 28 manually under the magnetic drive 30 in the conventional manner. In this case, the pressure sections 22 of the lever means 10 interact directly with the upwardly facing counterpressure surfaces of the stirrer shaft 28.

Before the stirrer shaft 28 is put into operation, the operator activates the closed-position securing mechanism by means of the securing pin 24a, so that during operation there is no risk that the stirrer shaft 28 and thus the entire bioreactor can be unintentionally released from the holding device 26 by accidentally pressing the lever 18 of the lever means 10.

Removal of the bioreactor from the holding device 26 is carried out in the reverse order of the steps described above. If a gripper 36 is used, it is mounted on the collar of the stirrer shaft 28. Before the lever 18 can be pressed down from the upper closed position to release the magnetic coupling, the closed-position securing mechanism must be deactivated by means of the securing pin 24a. By pushing the lever 18 down into the release position, the pressure sections 22 come into engagement with the counterpressure surfaces 40 of the gripper 36 and thus distribute the applied force; in addition, the stirrer shaft 28 is protected by avoiding direct contact with the pressure sections 22. The stirrer shaft 28 is in this way pushed down until the magnetic attraction forces are largely overcome and the magnetic coupling is released. This movement of the stirrer shaft 28 is guided, at least at the beginning. During this process, the operator can hold the bioreactor using the gripper 36 so that the bioreactor does not fall uncontrollably.

If no gripper 36 is used, the pressure sections 22 of the lever means 10 press directly on the counterpressure surfaces of the stirrer shaft 28. When the magnetic coupling is released, the operator holds the bioreactor in the usual way.

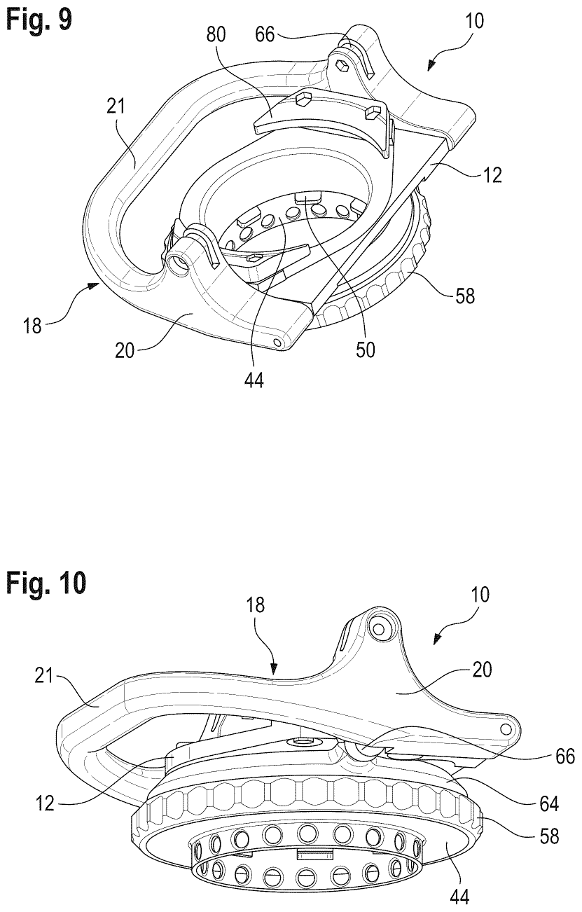

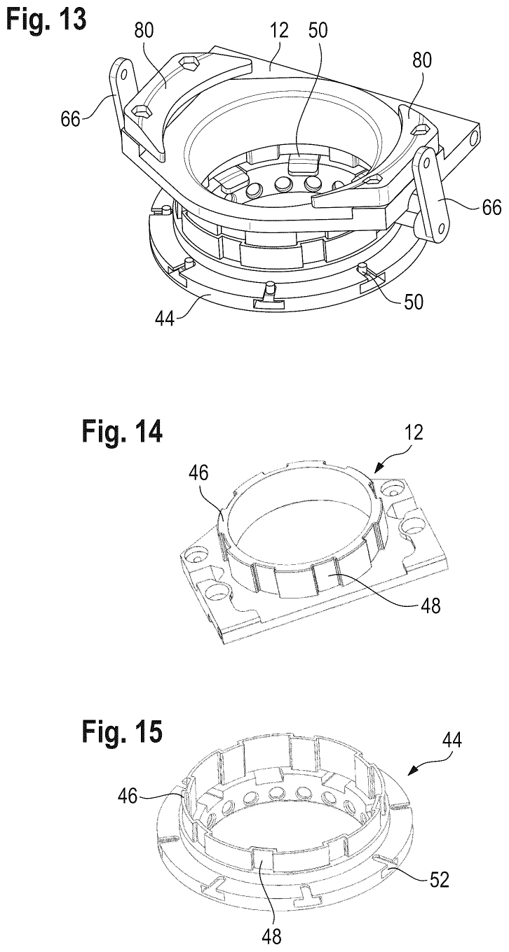

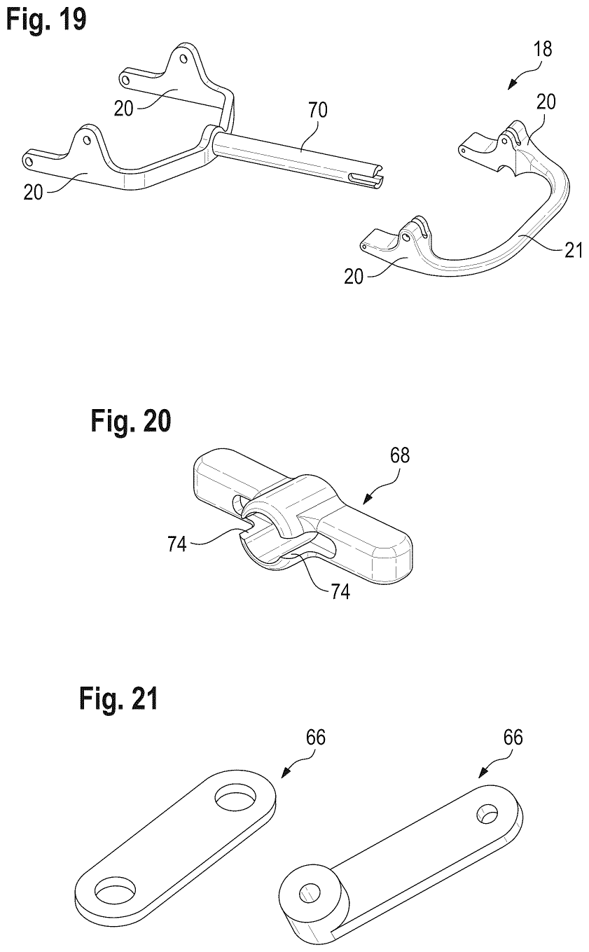

FIG. 9 and 10 or 11 and 12 show a first and a second variant of an alternative second embodiment of the lever means 10. Apart from the fact that the first variant has no closed-position securing mechanism, the two variants differ only in the design of the lever 18, which is designed in one piece in the first variant and in two pieces in the second variant (see FIGS. 19 and 20). The other essential components of the lever means 10, which are substantially identical in both variants of the second embodiment, are shown together in FIG. 13 (without the lever 18) and individually in FIGS. 14 to 18 and 21 to 24.

The same reference numerals are used for the components known from the first embodiment described above, and in this respect, reference is made to the above explanations. In the following, only the differences in the structure of the lever means 10 compared to the first embodiment will be discussed. The function and operation of the lever means 10 are substantially identical.

The adapter plate 12 (see FIG. 14) of the lever means 10 mounted on the holding device 26 is designed here such that it can receive a pressure ring 44 (see FIG. 15) fastened to the bioreactor or to the stirrer shaft 28. To this end, the adapter plate 12 and the pressure ring 46 each have a guide tube 46 and slot geometries 48 which are matched to each other. The guide tubes 46 and the slot geometries 48 prevent jamming or tilting when closing or releasing the magnetic coupling. In particular, it is thus ensured that when pressing on the upper flange of the stirrer shaft 28, the force for releasing the magnetic coupling is applied evenly.

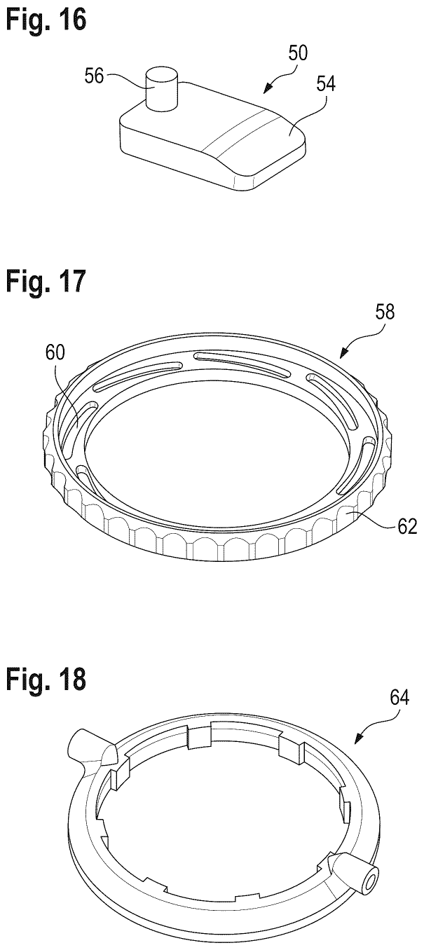

To secure the connection between the pressure ring 46 fastened to the bioreactor or the stirrer shaft 28 and the adapter plate 12 of the lever means 10 mounted on the holding device 26 during the running process, several slot nuts 50 (see FIG. 16) are provided, which can be inserted radially from the outside inwards into corresponding cutouts 52 of the pressure ring 46. The slot nuts 50 have clamping surfaces 54 and driving pins 56 which project axially when the slot nuts 50 are inserted into the cutouts 52. The slot nuts 50 are pushed so far inwards that the clamping effect on the clamping surfaces 54 is sufficiently large to ensure a secure connection.

The insertion and securing of the slot nuts 50 is carried out by means of a locking ring 58 (see FIG. 17), which has guide tracks 60 adapted to the position and shape of the drive pins 56, similar to a bayonet lock. The connection is secured by placing and turning the locking ring 58, wherein an adjustable torque limiter (not shown) can optionally be provided.

The adapter plate 12, the pressure ring 44, the slot nuts 50 and the locking ring 58 replace a Tri-Clamp connection, the manual handling being simplified not least by a profiled gripping surface 62 of the locking ring 58.

A supporting ring 64 (see FIG. 18) rests on the pressure ring 44, which is pressed down when the magnetic coupling is released and transmits the force required to overcome the magnetic force to the pressure ring 44 and thus to the stirrer shaft 18. This means that the supporting ring 64 corresponds functionally to the pressure sections 22 of the first embodiment. In addition, the supporting ring 64 serves to fix the locking ring 58.

FIG. 19 shows the pivot sections 20 of the lever means 10, which are rotatably mounted on the adapter plate 12. The pivot sections 20 and the connecting elements 66 between the lever 18 and the pressure ring 44, shown individually in FIG. 21, are used to convert a downward pivoting movement of the lever 18 into a downward linear movement of the supporting ring 64 and the pressure ring 44.

According to the second variant of the second embodiment, the gripping piece 68 shown in FIG. 20 is mounted on an extension arm 70.

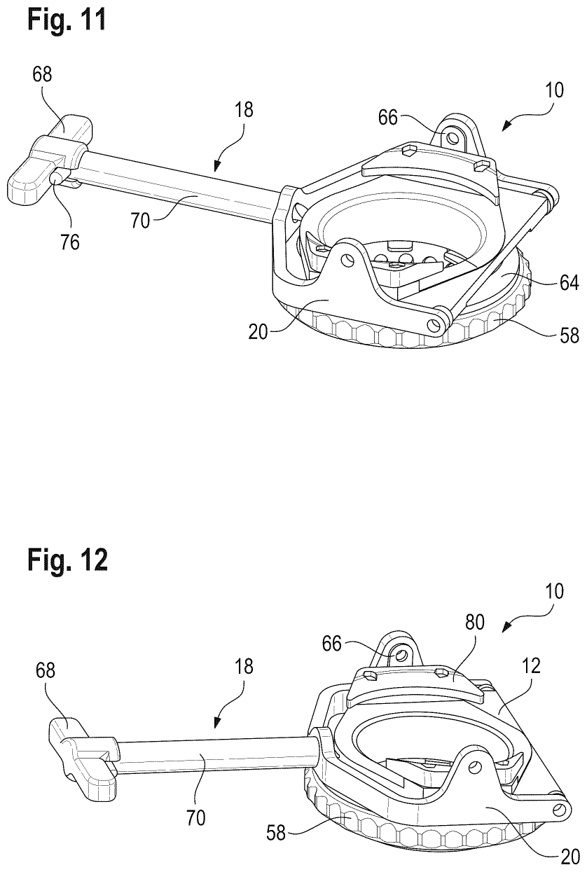

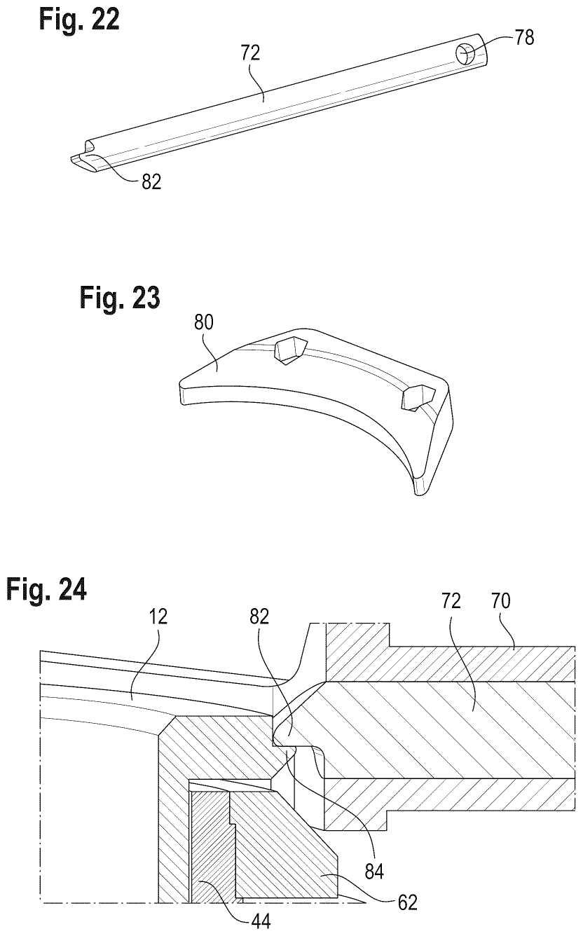

A cavity is formed in the extension arm 70 to receive a counterpart 72 of the adapter plate shown in FIG. 22, which is axially displaceable therein in the longitudinal direction against the force of a tension spring (not shown). In addition, recesses 74 for a release cross pin 76 (see FIG. 11) are formed in the extension arm 70. The release cross pin 76 can be inserted through a cross bore 78 of the adapter plate counterpart 72 so that an operator can pull the adapter plate counterpart 72 towards the gripping piece 68.

The gripping piece 68, the adapter plate counterpart 72 mounted in the extension arm 70 and the release cross pin 76 form here the locking-position securing mechanism.

FIG. 23 shows one of the two screw-on counter surfaces 80 for mounting the lever means 10, more precisely the adapter plate 12, on the holding device 26.

FIG. 24 shows the lever means 10 in the locked, upper closed position. The magnetic coupling is closed and the slot nuts 50 secure the stirrer shaft 28. The closed-position securing mechanism is activated as the adapter plate counterpart 72 is forced by spring force towards the adapter plate 12, thus maintaining engagement of a front engagement section 82 of the adapter plate counterpart 72 with a counter section 84 of the adapter plate 12, therefore preventing the lever 18 from being pushed down.

To release the stirrer shaft 28 from the magnetic drive 30 of the holding device 26, the operator must first deactivate the closed-position securing mechanism by pulling the adapter plate counterpart 72 against the spring force towards the gripping piece 68 of the lever by means of the release cross-pin 76. This disengages the adapter plate counterpart 72 from the counter section 84 of the adapter plate 12, so that the lever means 10 is unlocked. It is now possible to push the lever 18 down and thus release the magnetic coupling as already described above.

In the case of large bioreactors, it may be difficult to reach and manually operate the lever means 10. Therefore, an actuating means 86—only symbolically indicated in FIG. 8—may be provided on the lever means 10 (regardless of the specific design thereof), which allows remote operation of the lever means 10. In this case, the operator does not have to directly push or pull the handle 21 of the lever means 10 to pivot the latter from the release position into the locking position or vice versa, but can instead “remotely control” this using the actuating means 86.

The actuating means 86 may, for example, have a cable pull or a mechanical linkage. It is also possible that an electric, hydraulic or pneumatic drive is provided for the actuating means 86. In this case, the actuating means 86 can also be operated automatically as part of a (partially) automated process.

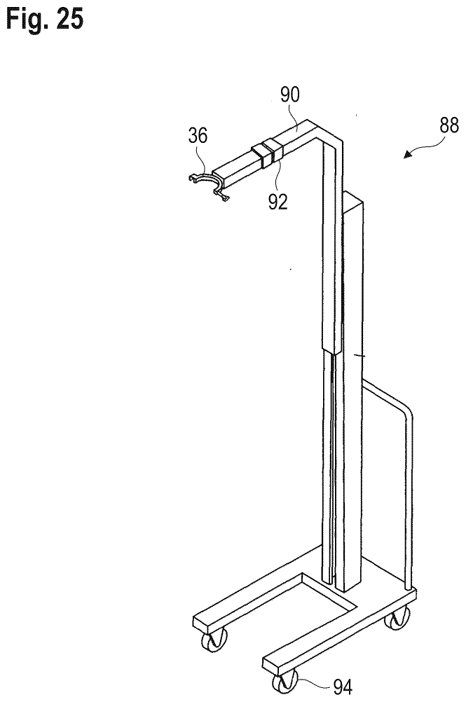

FIG. 25 shows a transport device 88 for lifting and transporting a container, in particular a bioreactor. The gripper 36 described above is fastened to a support arm 90 of the transport device 88. With such a transport device 88, it is possible, due to the gripper 36, to hold an unpacked bioreactor which includes a stirrer shaft 28 having a second coupling side 34 and to transport it to a final installation site, i.e. to a holding device 26 which includes a magnetic drive 30 having a first coupling side 32. The bioreactor is then mounted on the holding device 26 by means of the magnetic coupling as described above. After mounting, the transport device 88 can be removed from the stirrer shaft 28 using the gripper 36.

For easier handling of the bioreactor, the transport device 88 can have a rotating device 92, by means of which the gripper 36 can be rotated about a horizontal and/or vertical axis. The transport device 31 is also provided with wheels 94 and can thus be moved at least manually. However, it may also be provided that at least one of the wheels 94 can be driven by a motor.

In principle, it is also possible to provide the entire holding device-without the magnetic drive 30, but with a first coupling side 32—on the transport device 88. The bioreactor can then be mounted on the transport device 88 by means of the magnetic coupling as described above and dismounted accordingly after transport.

The components of the lever means 10 (including the gripper 36 of the first embodiment) are designed such that they can be manufactured at least for the most part by 3D printing (additive manufacturing process).

The invention described herein with reference to various example embodiments is applicable to any stirrer or connection which is magnetically coupled and decoupled, such as stirrers of disposable bioreactors made of rigid or flexible plastic material or of other mixing systems.

### LIST OF REFERENCE NUMERALS

10 lever means
12 adapter plate
14 opening
16 fastening sections
18 lever
20 pivot sections
21 handle
22 pressure sections
24a securing pin
24b securing bolt
25a upper bore
25b lower bore
26 holding device
28 stirrer shaft
30 magnetic drive
32 first coupling side
34 second coupling side
36 gripper
38 receiving section
40 counterpressure surfaces
42 gripping sections
44 pressure ring
46 guide tube
48 slot geometries
50 slot nuts
52 cutouts
54 clamping surfaces
56 driving pins
58 locking ring
60 guideways
62 gripping surface
64 supporting ring
66 connecting elements
68 gripping piece
70 extension arm
72 adapter plate counterpart
74 recesses
76 release cross pin
78 cross bore
80 screw-on counter surfaces
82 engagement section
84 counter section
86 actuating means
88 transport device
90 support arm
92 rotating device
94 wheels

1. A holding device for a container, comprising a magnetic drive for a separate stirrer shaft and a lever means mechanism for releasing the stirrer shaft from the magnetic drive, wherein the magnetic drive has a first coupling side and the stirrer shaft has a second coupling side of a magnetic coupling, which can be connected to the first coupling side, wherein the lever mechanism has at least one pressure section and is pivotable between a closed position and a release position, wherein, when the lever mechanism is pivoted into the release position, the at least one pressure section is moved downwards to engage directly or indirectly on the stirrer shaft.

2. The holding device according to claim 1, wherein the lever mechanism includes an adapter plate having a lever pivotally mounted thereon and a central opening which is located opposite the first coupling side and through which the second coupling side of the stirrer shaft projects in a mounted state thereof.

3. The holding device according to claim 2, wherein the lever mechanism is detachably fastened to the holding device by at least one fastening section.

4. The holding device according to claim 2, comprising a plurality of the at least one pressure sections arranged in a distributed manner.

5. The holding device according to claim 1, wherein the at least one pressure section is formed by a linearly guided sliding element which moves vertically downwards when the lever means mechanism is pivoted into the release position.

6. The holding device according to claim 2, wherein the at least one pressure section is formed by a linearly guided sliding element which moves vertically downwards when the lever mechanism is pivoted into the release position, and the sliding element is rotatably mounted on a pivot section of the lever mechanism and is guided in a recess of the adapter plate.

7. The holding device according to claim 1, wherein the lever mechanism comprises a closed-position securing mechanism which can be activated and deactivated, wherein the lever mechanism is fixed in the closed position when the closed-position securing mechanism is activated and is pivotable between the closed position and the release position when the closed-position securing mechanism is deactivated.

8. The holding device according to claim 1, wherein the lever mechanism has a release-position securing mechanism which can be activated and deactivated, wherein the lever mechanism is fixed in the release position when the release-position securing mechanism is activated and is pivotable between the release position and the closed position when the release-position securing mechanism is deactivated.

9. The holding device according to claim 1, further comprising a separate gripper shaped such that it can be mounted on a collar of the stirrer shaft, wherein the gripper includes at least one counterpressure section having a counterpressure surface which faces upwardly in a mounted state of the stirrer shaft and comes into engagement with the pressure section when the lever mechanism is pivoted into the release position.

10. The holding device according to claim 9, wherein the gripper has a receiving section having an inner diameter which corresponds approximately to an outer diameter of the first coupling side.

11. The holding device according to claim 10, wherein the gripper has at least one gripping section by which an operator can hold the gripper.

12. The holding device according to claim 11, wherein the gripper can be detachably mounted on the stirrer shaft.

13. The holding device according to claim 1, wherein at least part of the lever mechanism is manufactured by an additive manufacturing method.

14. An arrangement comprising the holding device according to claim 1 and the container, wherein the stirrer shaft is arranged in an interior of the container and has the second coupling side at its upper end, wherein the stirrer shaft is mounted on the magnetic drive of the holding device by closing the magnetic coupling such that the magnetic drive can set the stirrer shaft into a rotary motion.

15. The arrangement according to claim 14, wherein the container is a bioreactor.

16. The holding device according to claim 1, wherein the container is a bioreactor.

17. The holding device according to claim 1, comprising a plurality of the least one pressure sections arranged in a distributed manner.

18. The holding device according to claim 9, wherein the gripper has at least one gripping section by which an operator can hold the gripper.

19. The holding device according to claim 9, wherein the gripper can be detachably mounted on the stirrer shaft.
